(19) United States
(12) Patent Application Publication
(10) Pub. No.: US 2002/0068878 A1
(43) Pub. Date: Jun. 6, 2002

(54) Surgical instrument designed in particular for biopsies of the rectal mucosa

(76) Inventors: Vincenzo Jasonni, Genova (IT); Giuseppe Martucciello, Genova (IT); Alessio Pini Prato, Genova (IT)

Correspondence Address: YOUNG & THOMPSON, 745 SOUTH 23RD STREET 2ND FLOOR, ARLINGTON, VA, 22202

(21) Appl. No.: 09/975,928

(22) Filed: Oct. 15, 2001

(30) Foreign Application Priority Data
Oct. 13, 2000 (IT) MI2000A002211

(51) Int. Cl. A61B 10/00

(52) U.S. Cl. 600/566; 600/565

(57) ABSTRACT

Disclosed is a surgical instrument of the type comprising a probe which has a hole in the side wall and contains a knife in contact with the wall of said hole together with means designed to create a vacuum inside said probe, characterised in that said means designed to create a vacuum inside said probe are integrated into the instrument, which is also equipped with means designed to activate said vacuum-creating means and said knife simultaneously.

The present invention relates to an improved biopsy instrument, designed in particular for biopsies of the rectal mucosa, in which the means that develop the vacuum required for suction of the mucosa simultaneously activate the cutting tool so that the operator can take the sample with one hand, thus operating without the aid of an assistant.

The instrument is characterised by its particular practicality, as all operations can be performed by the operator merely by pulling a trigger; it is also equipped with means which can regulate the extent of the vacuum generated, and therefore the suction of the mucosa.

It is well known that enzymatic evaluation of the enteric nervous system (ENS) by acetylcholinesterase (AChE), NADPH-diaphorase and lactic dehydrogenase techniques is required to diagnose Hirchsprung's disease (congenital aganglionic megacolon).

This allows the submucosal ganglionic structures and any AChE-positive hypertrophic fibres to be identified and characterised.

A definitive diagnosis of Hirchsprung's disease, neurogenic achalasia of the anal sphincter and intestinal neuronal dysplasia (IND) can be formulated by the above mentioned techniques.

It is necessary to sample mucosa and submucosa in order to perform these histochemical tests.

A rectal biopsy instrument which operates by suction of the mucosa, comprising a probe or cannula with a substantially cylindrical cutting instrument sliding inside it, is already known for this purpose.

There is a hole in the side wall of the probe, and the inside of the probe is connected to an external suction device.

The vacuum created by said instrument in the probe sucks the mucosa through the hole, so that it partially protrudes into the probe.

The cutting tool is then made to slide in contact with the perforated wall and cut a small amount of rectal wall, which is then removed for subsequent analysis.

Although this known instrument allows acceptable biopsy tissue to be obtained, it has a number of drawbacks and limitations.

Primarily, it requires the presence of a physician who manoeuvres the instrument and an assistant who operates the suction in order to develop the vacuum required, thus increasing the cost of each operation.

In addition, as the vacuum is created manually, its extent cannot be precisely controlled; as the manoeuvre is empirical, it is consequently impossible to obtain biopsy tissue of constant dimensions and depth, thus making it practically impossible to achieve a replicable result, which is essential for correct histochemical evaluation of the biopsy tissue.

These known instruments are not adjustable enough to different patients, because as probe and blade have a fixed dimension; moreover, as the instrument cannot be dismantled, it is difficult to decontaminate and sterilise.

To obviate these drawbacks, the present invention provides an instrument for biopsies of the rectal wall in which the interior of the probe is connected to a cylinder, inside which slides a piston which in turn pulls the cutting tool.

Thus the necessary vacuum can be created in the probe, and the knife which cuts the mucosa can be activated, with a single operation.

This and other characteristics will appear clearly from the following detailed description, supplied by way of example but not of limitation, by reference to the annexed figures in which:

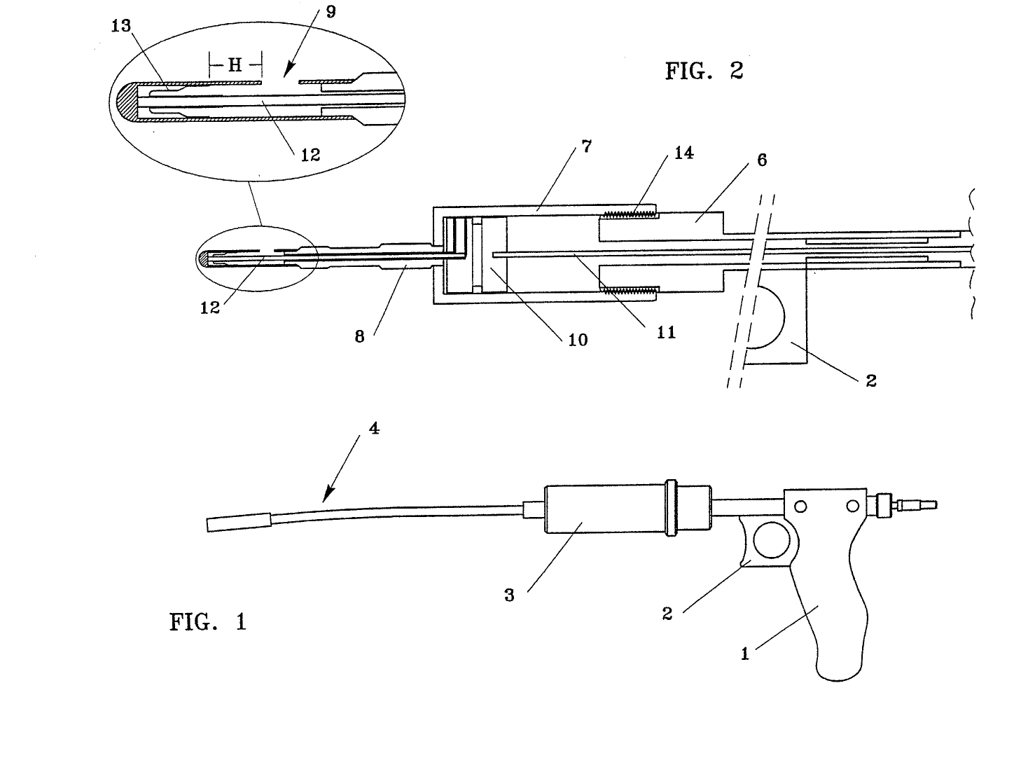

FIG. 1 illustrates a biopsy instrument in accordance with the invention;

FIG. 2 is a cross-section of the operational part of the instrument shown in FIG. 1.

As shown in FIG. 1, the instrument comprises a handle 1 with an operating trigger 2, to which the main instrument is fitted; the main instrument comprises a suction device 3 and a probe 4, which will be more particularly described hereafter.

As shown in FIG. 2, the suction device comprises a support 6 to which a cylinder 7 is fitted; a probe 4 is connected to the front of the said cylinder 7.

Probe 4 comprises a removable hollow rod 8 which communicates with the inside of cylinder 7, with a cap which has a hole 9 in the side wall near the tip.

Cylinder 7 is screwed to support 6, and can be fixed in various positions in relation to said support with a lock nut or the like.

This enables the inner volume of the cylinder to be varied.

A piston 10 integral with a rod 11 activated in a known way by trigger 2 slides inside the cylinder.

A rod 12 which pulls a knife 13 is fixed to the front of piston 10.

Knife 13 consists of a substantially cylindrical body which slides in the cap of probe 4 in contact with the inner wall of the probe, the length of said body being less than the distance between hole 9 and the tip of the probe.

The dimensions of the assembly are such that when piston 10 is in the forwardmost position in the direction of the probe, knife 13 is positioned against the tip of the probe, with the blade at a certain distance “H” from hole 9.

The volume of the zone between piston 10 and the cylinder wall to which rod 8 of probe 4 is fitted can be varied by screwing cylinder 7 to a greater or lesser extent onto threading 14 of support 6.

The device operates as follows.

When trigger 2 is operated, piston 10 is caused to move backwards in a known way, and tends to suck air from hole 9.

As the intestinal mucosa is lying against hole 9, the movement of the piston creates a vacuum in the probe, the value of which is a function of the piston stroke and the volume of the zone between the piston and the probe (according to the universal gas law PV=constant, so that when the volume increases as a result of the piston movement, the pressure is proportionally reduced, while the product remains constant).

The dimensions of the assembly are calculated to produce a maximum vacuum of approximately an atmosphere.

This vacuum tends to suck the mucosa through hole 9 so that it protrudes slightly into the probe.

As the movement of the piston that creates the vacuum also causes the movement of knife 13 via rod 12, when said knife reaches hole 9 it automatically slices a small amount of mucosa, thus allowing removal of the biopsy tissue.

With the embodiment of the invention, mere pressure on the trigger consequently generates the vacuum required for suction, and at the same time causes the knife to slide (sequentially) and slice the mucosa when it reaches the suction hole.

As a result, the operation can be completed in a single movement, using only one hand.

When the trigger is activated, piston 10 is caused to move backwards. During the first section of its travel (of length H), this creates a vacuum that sucks the mucosa through the hole, while the subsequent movement causes the knife to cut.

The extent of the vacuum created in the probe can be regulated merely by screwing cylinder 7 to support 6 to a greater or lesser extent.

This causes a variation in the volume of the zone between the piston and the probe; when this volume is increased, the effect of volume differential ΔV is reduced, as is pressure differential ΔP, thus producing a vacuum of the required value inside the probe.

As the thickness of the mucosa of the rectal wall is progressively greater in children, adolescents and adults, probes of different sizes in terms of hole measurement etc. can be made in order to suck in the correct amount of rectal mucosa and submucosa, thus preventing removal of too much or too little tissue.

All these characteristics, together with the possibility of replacing the probe, make the instrument ideal for performing biopsies on patients of any age. In addition, the fact that the various components can be dismantled allows the unit to be perfectly sterilised.

Although the above description specifically refers to the use of the instrument for rectal biopsies, the invention is obviously not limited to the case described, but could also be applied to the biomedical sector in general for endoscopic applications of all kinds.

Moreover, the characteristics of the instrument allow its use to be extended to other sectors, without departing from the scope of protection of the invention.

Different forms of embodiment could be included in the scope of the invention, and the dimensions or materials used could vary in accordance with practical requirements.

1. A surgical instrument of the type comprising a probe which has a hole in the side wall and contains a knife that slides in contact with the wall of said hole, together with means designed to create a vacuum in said probe, characterised in that said means designed to create a vacuum in said probe are integrated into the instrument, which is also fitted with means designed to activate said vacuum-creating means and said knife simultaneously.

2. An instrument as claimed in claim 1, characterised in that said means designed to create a vacuum inside said probe are connected to said cutting tool in such a way as to cut sequentially with the vacuum created.

3. An instrument as claimed in claim 2, characterised in that the probe is applied to a suction cylinder, with the interior of said probe communicating with said cylinder, and that a piston connected via a rod to the knife located in the probe slides inside said cylinder so that the first part of the piston travel creates a vacuum in the probe and moves the knife close to the hole in the probe wall, and the subsequent piston travel causes the knife to slice the tissue.

4. An instrument as claimed in claim 3, characterised in that said cylinder is fitted in such a way as to be adjustable on its support, so that the inner dimensions of the chamber between the piston and the probe can be varied.

5. An instrument as claimed in claim 4, characterised in that said cylinder is screwed to said support.

6. An instrument as claimed in any one of the preceding claims, characterised in that said probe is interchangeable.

7. An instrument for rectal mucosal biopsies, as claimed in any one of the preceding claims.
